(19) United States
(12) Patent Application Publication
(10) Pub. No.: US 2008/0317886 A1
(43) Pub. Date: Dec. 25, 2008

(54) Compositions for Preventing and Reducing Delayed Onset Muscle Soreness

(76) Inventor: Dennis R. Sparkman, Ennis, TX (US)

Correspondence Address: T. Ling Chwang;Jackson Walker L.L.P., 901 Main Street, Suite 6000, Dallas, TX, 75202, US

(21) Appl. No.: 12/084,416

(22) PCT Filed: Nov. 3, 2006
(86) PCT No.: PCT/US2006/043017

§ 371 Date: Jun. 5, 2008

Related U.S. Application Data
(60) Provisional application No. 60/733,272, filed on Nov. 3, 2005.

(51) Int. Cl. A61K 36/54; A61K 31/70; A61K 36/906; A61K 38/02; A61K 31/715

(52) U.S. Cl. 424/739; 514/23; 514/54; 424/756; 514/2; 514/58

(57) ABSTRACT

The present invention relates to the compositions that enhance post-exercise recovery processes to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquility immediately following exercise. The composition consists of any or all high-glycemic sugars and/or polysaccharides (e.g., sucrose, glucose, maltodextrin), all essential amino acids and beta-hydroxy-beta-methylbutyrate and can include other amino acids sources (e.g. whey protein), performance enhancing agents (e.g., caffeine, L-glutamate), anti-inflammatory agents (e.g., ginger, boswellia, curcumen), antioxidants (vitamin C, vitamin E, selenium, polyphenols,), insulin-mimicking agents (cinnamon, Banaba), analgesics (e.g. aspirin, ibuprofen, naproxen, acetaminophen), and to methods of treating humans and animals by administration of these novel compositions to humans and animals in need thereof.

This application claims priority to U.S. Provisional Patent Application Ser. No. 60/733,272, entitled “Compositions for Preventing and Reducing Delayed Onset Muscle Soreness,” filed on Nov. 3, 2005, the entire content of which is hereby incorporated by reference.

### BACKGROUND

This invention pertains to a composition that enhances post-exercise recovery processes in humans and animals to increase both strength and muscle mass. replace glycogen stores, and prevent inflammation, resulting in the preventing and reduction of delayed onset muscle soreness. Additionally, this composition provides the user with a feeling of muscle relaxation as well as mental tranquility immediately following exercise.

The muscle tissues of animals, including humans, are in a constant state of flux between the anabolic processes that build up muscle tissues and the catabolic processes, which degrades muscles tissues. A state of health exists when there is a balance between these two processes and derangements of the balance produce disease. Skeletal muscle comprises approximately 40 percent of the body while another 5 to 10 percent is smooth and cardiac. Skeletal muscle tissue is of particular importance for several reasons. First, it allows mobility by the lengthening and contraction of muscle fibers in addition to providing support to joints. Second, it provides strength and allows work to be preformed. Third, it enhances the metabolic rate by approximately 50 calories per day for each pound of muscle gained.

In order for muscles to maintain or gain strength, they must be exercised or used to a degree that is greater than what they are normally accustomed to. If one does the same exercises at the same pace every day, they will never become faster, stronger or have greater endurance. If one stops exercising when their muscles just start to burn from lactic acid production, they won't feel sore the next day and the muscles will not become stronger. All improvement in any muscle function comes from stressing and recovering. Stressing the muscles consists of exercising hard enough that the muscles begin to burn, due to lactic acid buildup, and reach the point of exhaustion or muscle failure. On the next day, the muscles feel sore because they have become damaged with resulting inflammation and need time to recover, which usually takes 3 to 4 days. This post-exercise soreness is called delayed onset muscle soreness (“DOMS”).

It takes approximately eight hours, more or less, to feel this type of soreness. When one finishes a hard workout, their muscles will feel exhausted, but as the day passes they will start to feel sore and by the next day the DOMS can be anywhere from moderate to severe, depending on the intensity of the previous day's workout. DOMS is caused by damage to the muscle fibers themselves. Muscle biopsies taken the day after intense exercise show bleeding and disruption of the z-band filaments that hold muscle fibers together as they slide over each other during a contraction. It can also consist of acute inflammation, accumulation of metabolites (e.g. free radicals) that increase damage, fluid retention, minor connective tissue tears and a combination of all of the above.

Scientists can determine how much muscle damage has occurred by measuring blood levels of a muscle enzyme called creatine phosphokinase (“CPK”). CPK is normally found in muscles and leaks out into the bloodstream when muscles are damaged. Those individuals who have the highest post-exercise blood levels of CPK will have the most muscle soreness.

It was once believed that DOMS was caused by a buildup of lactic acid in the muscles. Therefore, it was speculated that cooling down by exercising at a very slow pace after vigorous exercise would help prevent muscle soreness. This was not the case as cooling down only speeds up the removal of lactic acid from the muscles and it is now known that lactic acid has no effect on the degree of soreness, so cooling down slowly has no effect on preventing DOMS. Stretching does not prevent soreness either, since post-exercise soreness is not due to contracted muscle fibers.

DOMS has routinely been used as a guide to training, regardless of the sport or activity.

Depending on an individual's level of fitness, use of the body's muscles beyond one's normal limits, such as repetitive squatting while working in the garden, running in a marathon, or a workout using progressive resistance weight training will result in DOMS. Depending on how sore muscles are the following day, work or athletic training is usually curtailed for one to four days or only done in moderation at the very least. For this reason, it is recommended that one not train until the muscles have recovered and all muscle soreness has resolved. Therefore, marathon runners only run very hard and fast twice a week. Weightlifters only train the same muscle groups two times per week. The downside to DOMS is that it dictates a training schedule whereby two-thirds of the time is spent allowing muscles to recover. This amounts to a considerable amount of time where the athlete cannot train and is in a state of considerable discomfort.

Gains in athletic performance, such as strength and endurance, can only be accomplished by working out more often and at greater intensity. If post-workout recovery time could be shortened by the reduction of elimination of DOMS, athletes would be able to spend more time training and less down time while recovering.

When an individual begins pushing their muscles to the limit by exercising intensely for 45-60 minutes, the need for energy is enormous. During the first few minutes, the muscles burn up their existing energy stores of ATP, the molecule that provides the power for muscle contractions. The ATP can temporally be replaced by creatine phosphate, but then muscle tissue starts to burn glucose and glycogen for fuel. As the workout continues, blood insulin levels begin to drop and the liver starts to release more glucose into the blood. The low levels of insulin also cause the fat cells to release fatty acids, which can be burned as fuel. Essentially, the body is mobilizing its fuel reserves to support the intense use of its muscles.

Once the muscle activity ends, the body must begin to restore its depleted energy reserves and get blood glucose levels back to normal. After intense exercise, muscle glycogen stores can be completely depleted. To recover, one must take in nutrients, especially carbohydrates.

A prolonged state of intense exercise leaves the body in a hypoglycemic state, or with low levels of glucose. To replenish these levels, it is necessary to get carbohydrates back into the body as fast as possible. During the first 30 minutes after exercise, the body starts the recovery phase by mobilizing all its resources for replacing glycogen and building new muscle proteins (Sparkman, D. R., 1996a). If the nutrients are not immediately available, then these processes turn off and recovery will take a much longer time.

It has been shown that when a carbohydrate-rich supplement containing protein is given immediately after exercise, there is a rapid rise in the blood glucose levels, also known as hyperglycemia (Sparkman, D. R., 1994). This carbohydrate induced rise in blood insulin is greater than either carbohydrate or protein alone. Within 30 minutes of taking the carbohydrate supplement, the pancreas starts to release large amounts of insulin to get these high levels of blood glucose under control. The insulin binds to receptors on the muscle cells and facilitates the rapid entry of glucose and amino acids into the muscle cells.

These high plasma insulin levels will cause glucose to rapidly enter the muscles to the extent that it will again deplete blood glucose levels. The obvious benefit to these insulin surges is the anabolic effect of insulin on the muscle and its ability to increase the amounts of vital nutrients that enter the muscle cells. Glucose is taken in to restore muscle glycogen levels and amino acids are taken in to support protein synthesis. It would seem reasonable to think that when an individual rebounds into a state of hypoglycemia and insulin levels diminish, the body would enter into a catabolic state. However, it has been shown that the rapid rise and fall of blood glucose and insulin levels that are induced by a carbohydrate-protein supplement immediately after exercise causes a release of growth hormone five to six hours after exercise followed by a release in insulin-like grown factor that continues to promote protein synthesis. This is in addition to the testosterone that is released due to the lactate buildup in the blood from exercise.

During high-intensity exercise the concentrations of hormones in the blood and other body fluids can increase ten to twenty times over their levels at rest (Kraemer, W. J., 2000). At the same time that anabolic hormones are being released in response to exercise, catabolic hormones are also being released. Release of cortisol by the adrenal glands is directly linked to a variety of physiological functions, such as increased levels of proteolytic enzymes, inhibition of protein synthesis and the conversion of amino acids into carbohydrate. When muscle glycogen concentrations become low, blood cortisol levels increase to signal a need to shift to other energy substrates such as protein or fat so that judicious use is made of the little glucose that remains. While this is catabolic to muscles, the body is trying to preserve carbohydrate stores of glucose, which is the sole energy source for the brain. The body will literally cannibalize other tissues and organs to preserve glucose for the brain.

This degradation and loss of muscle tissue would result in a loss of strength, speed and mobility, which from an evolutionary standpoint would have a negative impact on an individual's ability to survive in a hunter-gatherer society where procurement of food depended on one's ability to work and search for food. The release of cortisol as a physiological survival response is obviously a double-edged sword in that it preserves the brain at the expense of muscle. To counter muscle loss, the body developed an additional response. During exercise, anabolic hormones, such as testosterone, insulin, insulin-like growth factors and growth hormone are released to help maintain muscle as well as bone and connective tissues.

Type I muscle fibers, or slow-twitch fibers, are generally fatigue resistant and have a high capacity for aerobic energy supply, but have limited potential for rapid force development. These fibers are best suited for aerobic work, such as running. Whereas endurance training does not provide a stimulus to increase the size of the Type I muscle fibers, these fibers do resist getting bigger with resistance training due to the need for optimal size for oxygen kinetics by down-regulating their testosterone receptors. At the same time, cortisol's influence is also diminished, both at the level of the receptor in the muscle and in the testes in men, which allows men to product more testosterone. This reduced influence of cortisol to promote protein degradation and influence of testosterone on protein synthesis results in hypertrophy of the Type I fibers in response to heavy resistance training. Therefore, type I muscle fibers gain more size by reducing the amount of protein degradation than by increasing the amount protein synthesis (Kraemer, W. J., 2000).

Type II muscle fibers, or fast-twitch fibers, are fatigable, have low aerobic power and rapid force development. These fibers are best suited for anaerobic work, such as weight lifting. Type II muscle fibers increase in size more by increasing the amount of protein synthesis than by reducing degradation, although both take place. The testosterone receptors in these fibers are up regulated by resistance training and therefore increase in size, whereas aerobic training has no effect. Other hormones, such as insulin, insulin-growth factor-1 and growth hormone also participate in the growth of muscle fibers in response to exercise. About 50 percent of muscle growth is thought to be due to growth hormone. Therefore, post-exercise nutritional supplementation that can support additional growth hormone release will greatly benefit gains in muscle mass and strength (Kraemer, W. J., 2000).

Because muscles are in a dynamic state of equilibrium whereby work or exercise causes the release of both catabolic and anabolic hormones, the net gain or loss of muscle mass depends upon the ratio of catabolic to anabolic processes. In order to gain more muscle mass in response to weight training, athletes have tried various nutritional strategies aimed at increasing protein syntheses and anabolic hormone release and/or decreasing catabolism and cortisol release.

As alluded to above, it was known that consuming a carbohydrate-rich meal immediately after exercise would raise insulin levels and begin a metabolic cascade of anabolic hormones that would increase muscle growth. In one study four groups of experienced bodybuilders were given post-workout supplements that consisted of: (1) 3.3 g carbohydrate (dextrose and maltodextrin) per pound of body weight, (2) 3 g protein (whey) per pound of body weight, (3) 2.3 g carbohydrates and 0.9 g protein per pound of body weight, and (4) water was given as a control (Sparkman, D. R., 1994; Chandler, R. M., et al., 1994). The supplements were given immediately after and two hours after exercise. Insulin levels were increased at 30 minutes with the carbohydrate-protein supplement giving the greatest increase, followed by the carbohydrate supplement, then the protein supplement. The two-hour supplements had little effect on the insulin response and no effect on blood glucose levels. The resistance training caused a release of growth hormone, which declined to baseline levels within two hours. However, at five to six hours post-exercise the growth hormone levels spiked again and showed a significant rise in the carbohydrate-protein and carbohydrate supplemented individuals. This second surge of growth hormone returned to baseline with two hours. Testosterone also increased as a result of exercise, but declined to baseline levels by six hours in all subjects.

Infusion of amino acids into resting, fasting human subjects increases amino acid transport into the muscle cell, stimulates muscle protein synthesis and improves net nitrogen balance from negative to slightly positive values, but had no effect on muscle protein breakdown (Biolo, G., et al., 1997). Resistance exercise also stimulates muscle protein synthesis; but muscle protein breakdown is also elevated so that, although net nitrogen balance increases, this balance remains negative (Biolo, G., 1995a). However, when an amino acid solution was infused into subjects after an hour of heavy resistance exercise, protein synthesis was increased to a greater extend than either exercise or the amino acid treatment alone (Biolo, G., 1997).

The body uses twenty amino acids for protein synthesis and of these twenty, nine are essential for man (Whitney, E. N & Rolfes, S. R., 2002) and ten are essential for animals (Jackson, et al., 2000) as they cannot be made by the body and must be obtained in the diet or by other external means. In some cases, nonessential amino acids can become conditionally essential when circumstances arise whereby the body cannot produce the amounts required and these amino acids must be supplemented from an external source. The conditionally essential amino acids are arginine, cysteine, glycine and tyrosine. Ingestion of nonessential amino acids is not necessary for stimulation of muscle protein synthesis (Tipton, K. D., 1999) because the body can quickly make them and therefore it is the essential amino acids, and sometimes the conditionally essential, that can limit muscle growth in response to exercise-induced protein synthesis.

Based on the knowledge that the post-exercise infusion of amino acids has a positive effect on protein synthesis (Biolo, G., et al., 1997) and that hyperinsulinemia stimulates amino acid uptake and protein synthesis (Biolo, G., et al., 1995b) by the muscles, an oral drink which consisted of 6 g essential amino acids and 35 g sucrose was tested for its effects on muscle protein synthesis. In a cross-over study, three healthy subjects were given either the essential amino acids with carbohydrate on one occasion followed by a placebo the next time, while the other three were given a placebo on one occasion followed by essential amino acids with carbohydrate on the next occasion. The supplements were given at one and three hours after resistance exercise. The essential amino acids/carbohydrate drink increased blood amino acids and insulin levels within 20 to 30 minutes when ingested at either one or three hours after resistance exercise. There was also an increase in net protein synthesis in the muscles within 20 minutes that returned to baseline within one hour. The placebo had no effect on insulin, amino acid levels or protein synthesis. Therefore, the essential amino acids/carbohydrate drink promoted anabolic conditions in the muscle beyond what could be achieved by exercise alone (Rasmussen, B. B., et al., 2000).

Although this increase in protein synthesis was transient, this was the highest protein synthetic rate observed under any circumstance and reflects a synergistic effect between the availability of essential amino acids, insulin and resistance training. The increase in muscle protein synthesis compared with resting values under various circumstances are as follows: (1) physiological hyperinsulinemia—50% (Biolo, G., et al., 1995b), (2) resistance exercise—100% (Biolo, g., et al. 1995b), (3) amino acid availability—150% (Biolo, G., et al., 1997b), (4) amino acid availability after resistance exercise—200% (Biolo, G., et al., 1997), (5) amino acid availability after resistance exercise with hyperinsulinemia—400% (Rasmussen, B. B., et al., 2000).

Since the early 1960's, leucine and its keto acid, alpha-ketoisocaproate (“KIC”) has been the subject of research into the regulation of muscle protein breakdown (Nair, K. S., et al., 2002; Frexes-Steed, M., et al., 1992). Leucine is unique in that it is not only an essential amino acid, but it is also a branched chain amino acid, which is catabolized during exercise. However, in the early 1990s, a downstream metabolite called beta-hydroxy-beta-methylbutyrate (“HMB”) was shown to have a positive effect on muscle protein (Nissen, S., et al., 1996; Nissen S., 2004).

In the body, HMB is produced in the muscle and liver from the amino acid leucine (Sabourin, P. J. and Bieber, L. L., 1981; Wagenmakers, A. J. M., et al., 1985). It is also obtained in trace amounts from foods, with plants having the lowest concentrations and meats having the highest concentrations (Nissen S., 2004). Although diet is a source of HMB, endogenous production of HMB from leucine generally far exceeds dietary intake. Therefore, foods containing large concentrations of leucine would probably have a greater influence on the circulating concentrations of HMB in the body. Studies where animals and humans have been given leucine intravenously have shown an increased rate of production and increased plasma levels of HMB (Zhang, Z., et al., 1993; Zachwieja, J. J., et al., 2004).

Synthetic HMB is available as a dietary supplement in the forms of calcium beta-hydroxy-beta-methylbutyrate or “CaHMB”, which has a molecular weight of 292 daltons. It is a white powder that is soluble in water. Absorption of HMB is rapid and plasma levels have been shown to increase in as little as 30 minutes.

The potential for HMB to be a beneficial dietary supplement to help increase strength and muscle mass in resistance training was quickly recognized by the bodybuilding community (Sparkman, D. R., 1997). A number of studies have investigated the effects of HMB on muscle damage following a single bout of strenuous exercise (Knitter, A. E., et al., 2000; Byrd, P., 1999). HMB supplementation has been shown to reduce the appearance of creatine phosphokinase (“CPK”) and lactate dehydrogenase (“LDH”), both indicators of muscle damage (Nissen, S., 2004). Both CPK and LDH are muscle enzymes that appear in the blood following muscle membrane damage and the amount in the blood is proportional to the severity of the damage. When runners were given either 3 g HMB per day or a placebo during six weeks of training followed by a 20 km run, blood samples taken after the run showed that the HMB-supplemented individuals had reduced levels of CPK and LDH compared to the placebo control subjects (Knitter, A. E., et al., 2000). Using a downhill running protocol, it was shown that supplementation with HMB reduced the amount of perceived muscle soreness and with less strength loss, suggesting an anti-catabolic effect on muscles which results in less damage (Byrd, P., et al., 1999).

Other studies have investigated the effects of HMB on muscle damage following an intense resistance-training program (Nissen S., et al., 1996; Kreider, R. B., et al., 1999; Panton, L. B., et al., 2000; Gallagher, P. M., et al., 2000; Jowko, E., et al., 2001). Men and women who took part in a 4-week weight training program and supplemented with HMB showed a 2% decrease in blood CPK levels while those using a placebo showed a 26% increase in CPK levels due to the weight training (Panton, L. B., et al., 2000). 3-methylhistidine (“3-MH”) is also a marker of muscle catabolism and when subjects undergoing intense resistance-weight training were supplemented with HMB there was a significant decrease in plasma 3-MH (Nissen S., et al., 1996). These findings suggest HMB supplementation minimizes the muscle damage that occurs from intense exercise.

In one study, plasma HMB levels were measured when HMB was administered with water or with 75 g glucose in water (Vukovich, M. D., et al., 2001). The results showed that both groups had identical blood glucose and insulin levels, but the group who consumed HMB with glucose had lower plasma HMB levels and less HMB excreted in the urine. Their conclusion was that the glucose slowed gastric emptying and prevented HMB from entering the blood as fast.

A recent study has described the use of HMB in the prevention of DOMS (van Someren, K. A., et al., 2005). In this study, eight males who had not exercised in a year were administered 3 g HMB/0.3 g KIC for 14 days prior to a single bout of exercise. After completing a muscle damaging exercise protocol of eccentric resistance training, it was determined that CPK and DOMS was significantly reduced after 14 days supplementation with HMB/KIC compared to controls. One previous study that looked at the effects of short term HMB supplementation on eccentric resistance training found that six days supplementation with 40 mg/kg HMB did not reduce DOMS after a single bout of exercise. Therefore, it would appear that prevention of DOMS by supplementation with HMB alone requires supplementation for more than 6 days prior to exercise.

It is also known that the essential amino acids have a very rapid half-life in the brain. Part of this rapid turnover is due to the essential amino acids being metabolic precursors that are converted into neurotransmitters. For example, tryptophan is converted into serotonin, which is responsible for relaxation. Phenylalanine is converted into dopamine and then into norepinephrine, which is responsible for pleasure and well being, respectively. Norepinephrine can protect endorphins, which are responsible for the runner's “high” that is experienced during prolonged exercise. Methionine is converted into S-adensylmethionine, which acts as a natural anti-depressant.

The central nervous system contains both insulin and insulin receptors. Insulin has been shown to facilitate the entry of amino acids across the blood brain barrier into the brain and has been suggested to be a neuromodulator. Additionally, amino acid transport into the brain is also controlled by the serum concentration of amino acids. Studies in which subjects were given diets devoid of certain amino acids have been able to demonstrate a negative mood change as a result. It is also known that foods, such as carbohydrates and protein (e.g. amino acids) can have a positive influence on mood. Dietary supplementation with tryptophan has been shown to be of benefit for sleep disorders, seasonal affective disorders and depression. S-adensylmethionine supplementation has been used for depression. Studies have reported that supplementation with phenylalanine and tyrosine can improve mood. Therefore, it is possible to modulate mood by nutritional means.

### SUMMARY

The present invention provides novel compositions and methods for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise.

This invention includes a high glycemic sugar along with essential amino acids to elicit a rise in insulin to drive the essential amino acids, beta-hydroxy-beta-methylbutyrate and glucose into the muscles to increase the anabolic response, decrease the catabolic response and replace glycogen, respectively, following exercise. These mechanisms act synergistically by increasing the gross anabolic response while simultaneously decreasing gross catabolism, which results in a net gain in protein synthesis and muscle growth and strength.

The increase in insulin by the high glycemic sugar and essential amino acids also increases the transport of essential amino acids across the blood brain barrier. This increased pool of essential amino acids provides the brain with a higher concentration of metabolic precursors to drive the synthesis of neurotransmitters. This increase in neurotransmitters that are capable of effecting mood is believed to be partly responsible for the feeling of tranquillity immediately following exercise.

The present invention overcomes another disadvantage and drawback of prior art, which is delayed onset muscle soreness that results after intense exercise. For a number of years, the health tread has been to follow low-carbohydrate diets (Atkins diet) and low glycemic diets (South Beach diet), which avoid either all carbohydrates altogether or all high-glycemic sugars and carbohydrates that can raise insulin levels, respectively. For this reason, most sport supplements avoid the use of sugar as a sweetener and rely on artificial sweeteners, such as sucralose, aspartame, or saccharine. HMB is usually provided as capsules, however, some preparations employ HMB and creatine as a drink mix, but the mixes are sweetened with artificial sweeteners. Even if some form of sugar was ever used in such a preparation; the function would be for taste, rather than function as no previous art has reported using high glycemic sugars to elicit an insulin response to drive HMB into the muscle to increase its efficacy.

Studies by Vukovich, et al (2001) indicates that when HMB is taken with high glycemic sugars, such as glucose, there is a reduction in absorption of HMB and lower plasma HMB levels. However, this composition of high-glycemic sugars and/or carbohydrates, essential amino acids and beta-hydroxy-beta-methylbutyrate immediately prevents or greatly reduces delayed onset muscle soreness following exercise. Without wanting to be bound by theory, the mechanism of action is believed to be that because HMB is a leucine amino acid metabolite, the insulin response caused by the sugar helps rapidly drive the HMB into the muscles. Vukovich, et al (2001) was not able to demonstrate that HMB remained unabsorbed and it is therefore our proposal that plasma HMB levels were lower in the presence of glucose because the insulin facilitated the entry of HMB into the muscle thus clearing it more rapidly from the blood. This accelerates the effects of the HMB, which causes a greater reduction in catabolism than HMB taken alone, while the essential amino acids elicits a concomitant anabolic response, resulting in a prevention of DOMS due to synergy.

The rapid transport of the essential amino acids and HMB into the muscle, and brain, results in enhanced post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise.

Inclusion of essential amino acids and beta-hydroxy-beta-methylbutyrate following exercise can still act synergistically to increase the net anabolic response by preventing muscle protein breakdown and supporting muscle protein synthesis to promote muscle growth in the absence of a high glycemic sugar, however the rapid delivery into the muscles, as well as transport across the blood brain barrier, would be slower due to the lack of a hyperinsulinemia response. Although this would not be the composition of choice for normal, healthy subjects, it would be applicable to diabetics who could not tolerate a high glycemic load due to their inability to product insulin or insulin resistance.

Therefore one aspect of the present invention is to provide novel compositions of any or all high-glycemic sugars and/or carbohydrates, any or all forms of essential amino acids and any or all salts of beta-hydroxy-beta-methylbutyrate for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise.

Another object of the invention is to provide novel compositions of any or all high-glycemic sugars and/or carbohydrates, any or all forms of essential amino acids, any or all salts of beta-hydroxy-beta-methylbutyrate and protein for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise.

It is another object of the invention to provide novel compositions of any or all high-glycemic sugars and/or carbohydrates, any and all forms of essential amino acids, any and all salts of beta-hydroxy-beta-methylbutyrate and performance enhancing agents for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise and helps prevent overtraining.

It is still another object of the invention to provide novel compositions of any or all high-glycemic sugars and/or carbohydrates, any or all forms of essential amino acids, any or all salts of beta-hydroxy-beta-methylbutyrate and anti-inflammatory agents for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise and can prevent additional soreness that might result from inflammation joints and ligaments.

It is still another object of the invention to provide novel compositions of any or all high-glycemic sugars and/or carbohydrates, any and all forms of essential amino acids, any and all salts of beta-hydroxy-beta-methylbutyrate and antioxidant agents for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise and can prevent free radical formation that may cause oxidative damage muscle tissues.

It is a further object of the invention to provide novel compositions of any or all high-glycemic sugars and/or carbohydrates, any and all forms of essential amino acids, any and all salts of beta-hydroxy-beta-methylbutyrate and insulin-mimicking agents for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise and helps augment insulin's ability to drive nutrients into target tissues.

It is a further object of the invention to provide novel compositions of any or all high-glycemic sugars and/or carbohydrates, any and all forms of essential amino acids, any and all salts of beta-hydroxy-beta-methylbutyrate and analgesic agents for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise and can prevent pain that might result from overuse of joints and ligaments.

It is a further object of the invention to provide novel compositions of any and all forms of essential amino acids and any and all salts of beta-hydroxy-beta-methylbutyrate for enhancing post-exercise recovery processes in humans and animals to increase both strength and muscle mass and prevent inflammation, resulting in the prevention and/or reduction of delayed onset muscle soreness. This composition could include one or more of the above performance enhancing agents, such as the inclusion of protein, performance enhancing agents, anti-inflammatory agents, antioxidants, insulin-mimicking agents and analgesic agents for the purposes described above.

It is a further object of the invention to provide novel compositions of any and all high-glycemic sugars and/or carbohydrates and any all salts of beta-hydroxy-beta-methylbutyrate for enhancing post-exercise recovery processes in humans and animals to increase both strength and muscle mass, replace glycogen stores, and prevent inflammation, resulting in the prevention and/or reduction of delayed onset muscle soreness. This composition could include one or more of the above performance enhancing agents, such as the inclusion of protein, performance enhancing agents, anti-inflammatory agents, antioxidants, insulin-mimicking agents and analgesic agents for the purposes described above.

It is a further object of this invention to provide novel tandem compositions, whereby any and all high-glycemic sugars and/or carbohydrates and any and all salts of beta-hydroxy-beta-methylbutyrate could be administered at one point in time followed by any and all high-glycemic sugars and/or carbohydrates and any and all forms of essential amino acids at another point in time that allows the ingredients to act synergistically in the body for enhancing post-exercise recovery processes in humans and animals to increase both strength and muscle mass, replace glycogen stores, and prevent inflammation, resulting in the prevention and/or reduction of delayed onset muscle soreness. Additionally, it provides a feeling of muscle relaxation as well as a feeling of mental tranquillity immediately following exercise. Either or both tandem compositions could include one or more of the above performance enhancing agents, such as the inclusion of protein, performance enhancing agents, anti-inflammatory agents, antioxidants, insulin-mimicking agents and analgesic agents for the purposes described above.

It is a further object of this invention to provide novel tandem compositions, whereby any and all salts of beta-hydroxy-beta-methylbutyrate could be administered at one point in time followed by any and all forms of essential amino acids at another point in time that allows the ingredients to act synergistically in the body for enhancing post-exercise recovery processes in humans and animals to increase both strength and muscle mass and prevent inflammation, resulting in the prevention and/or reduction of delayed onset muscle soreness. Either or both tandem composition could include one or more of the above performance enhancing agents, such as the inclusion of protein, performance enhancing agents, anti-inflammatory agents, antioxidants, insulin-mimicking agents and analgesic agents for the purposes described above.

### DETAILED DESCRIPTION OF PREFERRED EMBODIMENTS

The present invention relates to the compositions that enhance the post-exercise recovery process, in humans and animals to increase both strength and muscle mass, replace glycogen stores, and prevent inflammation, resulting in prevention and/or reduction of delayed onset muscle soreness. Additionally, it provides a feeling of muscle relaxation as well as a feeling of tranquillity immediately following exercise. The composition comprises any or all high-glycemic carbohydrates, such as sugars and/or polysaccharides (e.g., sucrose, glucose, maltodextrin), as well as any and all forms essential amino acids and any and all salts of beta-hydroxy-beta-methylbutyrate (“HMB”). The composition may also comprise any and all high-glycemic carbohydrates and any and all salts of HMB, without the essential amino acids being directly added or it may comprise any and all forms of essential amino acids and any and all salts of HMB without the high glycemic sugars being directly added. The composition my also consist of any and all high-glycemic carbohydrates and any and all salts of HMB followed in tandem by any and all high-glycemic carbohydrates and any and all forms essential amino acids. The composition my also consist of any and all salts of HMB followed in tandem by any and all forms essential amino acids. Optionally, these compositions may further comprise other amino acid sources (e.g. whey protein), performance enhancing agents (e.g., caffeine, L-glutamate, L-arginine), anti-inflammatory agents (e.g., ginger, boswellia, curcumen), antioxidants (vitamin C, vitamin E, selenium, polyphenols, fruit extracts), insulin-mimicking agents (cinnamon) and analgesics (e.g. aspirin, ibuprofen, naproxen, acetaminophen).

One aspect of the present invention pertains to a composition for preventing or reducing delayed onset muscle soreness comprising one or more high glycemic carbohydrates, one or more essential amino acids, and beta-hydroxy-beta-methylbutyrate (“HMB”). A further aspect of the present invention pertains to a composition for preventing or reducing delayed onset muscle soreness comprising one or more high glycemic carbohydrates and HMB. One further aspect of the invention pertains to a composition for preventing delayed onset muscle soreness comprising one or more essential amino acids and HMB. One further aspect of the invention pertains to a composition for preventing delayed onset muscle soreness comprising one or more high glycemic carbohydrates and HMB followed in tandem with one or more high glycemic carbohydrates and essential amino acids. One further aspect of the invention pertains to a composition for preventing delayed onset muscle soreness comprising HMB followed in tandem with essential amino acids. Another aspect of the present invention pertains to methods of treating humans and animals by administration of these novel compositions to humans and animals in need thereof.

High-glycemic sugars and/or polysaccharides are carbohydrates that are quickly digested and absorbed in the stomach. This causes a rapid rise in blood glucose levels, which results in the pancreas releasing insulin to drive glucose into the liver and muscles to restore normal blood glucose levels. In addition, insulin also causes (1) active transport of amino acids into cells, especially valine, leucine, isoleucine, tyrosine and phenylalanine, (2) increases the translations of messenger ribonucleic acids (mRNA) to form new proteins, (3) increases the transcription of deoxyribonucleic acid to form more mRNA, (4) inhibits the catabolism of proteins, and (5) suppresses gluconeogenesis, which uses amino acids as substrates—thus preserving amino acid pools (Guyton, A. C., 1981). Examples of high glycemic sugars are glucose (dextrose), maltose, sucrose, molasses, dehydrated cane syrup, maple syrup, fruit juices and some honeys. Examples of high-glycemic polysaccharides are maltodextrin, starches and flours (Brand-Miller, J., et al., 2003).

The essential amino acids are those that cannot be synthesized by the body and must be obtained from the diet (Whitney, E. N. and Rolfes, S. R., 2002). Because the body cannot make them, they can become rate limited when the body is in an anabolic state and producing muscle protein. The essential amino acids in humans are L-histidine, L-isoleucine, L-leucine, L-lysine, L-methionine, L-phenylalanine, L-threonine, L-tryptophan, and L-valine (Whitney, E. N, and Rolfes, S. R., 2002). The essential amino acids for all other vertebrates are L-arginine, L-histidine, L-isoleucine, L-leucine, L-lysine, L-methionine, L-phenylalanine, L-threonine, L-tryptophan, and L-valine (Jackson, N. S., et al., 2000). Alternatively, one could use racemic amino acids: D, L-arginine, D, L-histidine, D, L-isoleucine, D, L-leucine, D, L-lysine, D, L-methionine, D, L-phenylalanine, D, L-threonine, D, L-tryptophan, and D, L-valine at double the concentration.

Although the D-isomers of amino acids are not incorporated into proteins, the body uses them as metabolic precursors. The brain is known to use D-isomers of amino acids for neurotransmitter synthesis. Therefore, D-arginine, D-histidine, D-isoleucine, D-leucine, D-lysine, D-methionine, D-phenylalanine, D-threonine. D-tryptophan, and D-valine may be included along with the L-isomers.

Beta-hydroxy-beta-methylbutyrate (“HMB”) is a downstream metabolite of the essential amino acid leucine. The exact mechanism of action of HMB is not known, but it is believed that HMB works by improving cell membrane integrity by supplying adequate substrate for cholesterol synthesis. HMB is converted to HMG-CoA in the cytosol, which can be used for cholesterol synthesis in the cells. Cholesterol is needed for the synthesis of new cell membranes as well as the repair of damaged membranes. Certain cells, such as muscle cells, require de novo synthesis of cholesterol for proper cellular function. During periods of increased stress on muscle cells during intense exercise, the demand for cholesterol for growth and repair of cellular membranes may exceed what can be made through normal production from available endogenous HMG-CoA. Therefore, supplemental HMB may help meet this increased demand by supplying extra HMG-CoA for cholesterol synthesis. This cholesterol can then use used to stabilized muscle cell membranes after intense exercise.

Without wanting to be bound by theory, it is also postulated that HMB somehow directly decreases muscle proteolysis or protein breakdown by having a direct effect on transcriptional or translational control of genes, enzyme activities or other processes involved with proteolysis.

The forms of HMB could include but are not limited to β-hydroxy-β-methylbutanoic acid, calcium β-hydroxy-β-methylbutyrate, β-hydroxy-β-methylbutyrate amino acid salt, and tricreatine β-hydroxy-β-methylbutyrate. The immediate upstream precursor of HMB is α-ketoisocaproate, which is formed from the amino acid leucine. Therefore, α-ketoisocaproate and/or leucine could be used instead of HMB or used in conjunction with HMB. Other forms of α-ketoisocaproate, such as arginine-ketoisocaproate could also be used.

Protein is composed of the 20 essential and nonessential amino acids. When digested in the digestive tract, it provides a source of free amino acids for protein synthesis. Alternatively, a protein hydrolysate, which has been pre-hydrolyzed, can be used to provide a source of free amino acids that are readily available for absorption and transport into muscle tissue for protein synthesis. More importantly, it has been shown that when carbohydrate and protein are provided at a 2.5:1 ratio, respectively, there is a greater release of insulin than when either carbohydrate or protein are fed individually. Therefore, intact, partially hydrolyzed or completely hydrolyzed protein could be added to the invention to provide the sole source of essential amino acids or be added in addition to the free-form essential amino acids. In either case, as the carbohydrate-protein ratios approach 2.5:1, there would be a synergistic induction of insulin. Such protein sources might include one or a combination of, but would not be limited to, whey protein, soy protein, egg white protein, milk caseinate protein, meat extract protein, royal jelly and any polypeptide containing one or more of the 20 amino acids that make up biological proteins.

The current composition may optionally further comprise one or more performance enhancing agents, such as caffeine. Caffeine is a natural stimulate found in foods and is the most widely consumed psychoactive drug in the world. It belongs to a group of lipid-soluble compounds called methylxanthines. Physiologically, caffeine stimulates the nervous system, which in turn causes the release of epinephrine from the adrenal medulla. It also increases the body's heart rate and peripheral vasodilatation. At the cellular level, consumption of caffeine includes an increased release of calcium from the muscle sarcoplasmic reticulum and elevation of intracellular cyclic adenosine monophosphate, which is responsible for the activation of hormone sensitive lipase that results in the mobilization of fatty acids from fat cells. Caffeine can also block adenosine receptors, which is thought to explain its stimulating effect as adenosine has a calming effect. It also acts as a mild diuretic.

Mounting evidence indicates that ingestion of caffeine improves aerobic endurance by increasing time to exhaustion, speed and power output. A recent study suggests that caffeine can increase performance by increasing carbohydrate uptake by the body. Therefore, if caffeine can increase the absorption of high-glycemic sugars and/or carbohydrates, it will potentiate the insulin response to further facilitate the uptake of the essential amino acids and HMB by the muscles. The most obvious sources of caffeine would be USP caffeine, but other sources of caffeine could be herbs or herbal extracts. Such herbs or extracts could include, but is not limited to coffee, teas, or guarana.

The current composition may also further comprise L-glutamine. L-glutamine is a nonessential amino acid. However, it should be considered a conditionally essential amino acid because during times of stress, the normal endogenous synthetic pathways may not meet the body's need for glutamine. Glutamine is the most abundant amino acid in the plasma and skeletal muscles. It accounts for 60% of the total intramuscular pool of free amino acids. Research does not support a role for glutamine in strength or endurance training. More attention has been directed toward the potential use of glutamine to boost the immune system of athletes.

It is known that glutamine can become depleted during times of severe stress, such as infection or injury (Sparkman, D. R., 1996b). The body can react to prolonged exercise and overtraining as a stressful event. Intense and prolonged exercise can cause damage to muscle tissue, which can also be perceived by the body as an injury. During this training the use of glutamine by other organs increases in response to bodily stress. As a result, plasma glutamine levels begin to plummet drastically. To replenish these levels, the muscles start to release their glutamine stores into the blood. Intense exercise can also cause the release of catabolic hormones, such as cortisol, which further depletes muscles of glutamine.

Overtraining results when increasing volumes and intensity of training is out of balance with recovery time. One factor that may contribute to overtraining syndrome is continual daily exercise, which may deplete the muscles of glutamine to the point they can no longer recover. Over time this constant drain of glutamine reserves without ample recovery time may damage the muscle's glutamine synthesizing system.

Because glutamine is the primary source of fuel for cells of the immune system, particularly lymphocytes, macrophages and killer cells, individuals suffering from overtraining are more susceptible to disease and infections. During times of immunological assaults, the requirement for glutamine increases in order to support the rapid cell division, protein synthesis and the production of antibodies and cytokines. Thus, low glutamine levels can impair the immune system. Because the current compositions will reduce muscle soreness and shorten the recovery time needed between training, addition of glutamine may be beneficial to prevent overtraining syndrome.

The composition may also further comprise L-arginine. L-arginine is a conditionally essential amino acid that can become depleted in times of stress. It is known to have both anabolic and immunomodulatory properties and a deficiency due to post-exercise stress could have a negative impact on muscle repair and immune function. L-arginine and L-lysine are two of the most potent stimulators of insulin of all the amino acids (Guyton, A. C., 1981). L-arginine is a substrate for nitric oxide synthase enzyme, which converts arginine to nitric oxide. Nitric oxide causes vasodilatation, which can increase blood flow to the muscle to deliver nutrients and remove waste.

The composition may also further comprise one or more anti-inflammatory agents. Ligaments and tendons can become inflamed during intense exercise. A number of herbal compounds have been found to possess anti-inflammatory properties. Such herbs or extracts thereof could include, but is not limited to, ginger (Zingiber officinale), boswellin (Bosvellia serrata), and curcumen (Curcuma longa).

The composition may also further comprise one or more anti-oxidant agents. During exercise, the bodily consumption of oxygen is increased 10- to 15-fold greater than resting levels. Oxygen uptake in the active skeletal muscle may increase up to 100-fold. If the same percent of reactive oxygen species (free radicals) holds true, exercise should lead to a large increase in total body free radical production and an even larger increase in the working muscles. This excessive production of free radicals in muscle tissue could lead to muscle oxidative damage, which could lead to impaired contractibility. Muscle tissue is lower in many enzymes that prevent free radical formation, such as superoxide dismutase, catalase, glutathione, and glutathione peroxidase. Studies have shown that animals given green tea daily showed no change in oxidative stress biomarkers after exercise whereas those given water showed a 290% increase in oxidative stress biomarkers. Inclusion of one or more antioxidants or compounds exhibiting antioxidant activity should quench free radicals formed during exercise and help prevent oxidative damage to the body, especially the muscles. Such antioxidants could include one or more of the following: vitamin A, beta-carotene, vitamin C, vitamin E, selenium, α-lipoic acid, glutathione, superoxide dismutase, polyphenols, anthrocyanins, carotenoids, astaxanthin, and fruit extracts. These examples of antioxidants and compounds with antioxidant-like properties are not all-inclusive, but merely represent examples of the many known and yet to be discovered compounds to which one skilled in the art could choose to incorporate into this invention.

The composition may also further comprise one or more insulin-mimicking agents or modulating agents to facilitate delivery of nutrients to the muscles. Cinnamon is known to contain a water-soluble polyphenol compound called methylhydroxychalcone polymer (MHCP). MHCP has been shown to lower blood glucose levels; stimulate glucose uptake and glycogen synthesis. Therefore, cinnamon or an aqueous extract containing MHCP would enhance nutrient uptake in tissues as well as prove beneficial to type 2 diabetics. Banaba (Lagerstroemia speciosa) contains colosolic acid which has also been shown to lower blood sugar in type 2 diabetics.

The composition may also further comprise one or more analgesic agents. Although this invention prevents or reduces delayed onset muscle soreness, exercise also causes stress on other body parts such as joints, ligaments, and tendons, which can cause pain and discomfort following exercise. For this reason, the incorporation of an analgesic into this invention could help alleviate pain in other tissues not directly affected by this invention. Such analgesics could include aspirin, ibuprofen, naproxen, acetaminophen or any other pharmacological compound designed to reduce pain.

Table 1 below shows some possible combinations of the components described above, which are combined to form the compositions of the present invention.

TABLE 1

| | | | | | | | Antioxidants | |
|---|---|---|---|---|---|---|---|---|
| | High Glycemic | Essential | | Intact or | Performance | Anti- | and/or | |
| Formula | Sugars and/or | Amino | | Hydrolyzed | Enhancing | inflammatory | antioxidant-like | |
| Number | Carbohydrates | Acids | CaHMB | Protein | Agents | Agents | compounds | Analgesics |
| 1 | X | X | X | | | | | |
| 2 | X | X | X | X | | | | |
| 3 | X | X | X | X | X | | | |
| 4 | X | X | X | X | X | X | | |
| 5 | X | X | X | X | X | X | X | |
| 6 | X | X | X | X | X | X | X | X |
| 7 | X | X | X | | X | | | |
| 8 | X | X | X | | X | X | | |
| 9 | X | X | X | | X | X | X | |
| 10 | X | X | X | | X | X | X | X |
| 11 | X | X | X | | | X | | |
| 12 | X | X | X | | | X | X | |
| 13 | X | X | X | | | X | X | X |
| 14 | X | X | X | X | | X | X | X |
| 15 | X | X | X | | | | X | |
| 16 | X | X | X | | | | X | X |
| 17 | X | X | X | | X | | X | X |
| 18 | X | X | X | X | | | X | X |
| 19 | X | X | X | | | | | X |
| 20 | X | X | X | | | X | | X |
| 21 | X | X | X | | X | | | X |
| 22 | X | X | X | X | | | | X |

The efficacy of HMB is enhanced in the presence of high-glycemic sugars and/or carbohydrates. Table 2 below shows some possible combinations of the components described above, not including the essential amino acids.

TABLE 2

| | | | | | | Antioxidants | |
|---|---|---|---|---|---|---|---|
| | High Glycemic | | Intact or | Performance | Anti- | and/or | |
| Formula | Sugars and/or | | Hydrolyzed | Enhancing | inflammatory | antioxidant-like | |
| Number | Carbohydrates | CaHMB | Protein | Agents | Agents | compounds | Analgesics |
| 1 | X | X | | | | | |
| 2 | X | X | X | | | | |
| 3 | X | X | X | X | | | |
| 4 | X | X | X | X | X | | |
| 5 | X | X | X | X | X | X | |
| 6 | X | X | X | X | X | X | X |
| 7 | X | X | | X | | | |
| 8 | X | X | | X | X | | |
| 9 | X | X | | X | X | X | |
| 10 | X | X | | X | X | X | X |
| 11 | X | X | | | X | | |
| 12 | X | X | | | X | X | |
| 13 | X | X | | | X | X | X |
| 14 | X | X | X | | X | X | X |
| 15 | X | X | | | | X | |
| 16 | X | X | | | | X | X |
| 17 | X | X | | X | | X | X |
| 18 | X | X | X | | | X | X |
| 19 | X | X | | | | | X |
| 20 | X | X | | | X | | X |
| 21 | X | X | | X | | | X |
| 22 | X | X | X | | | | X |

The composition can consist of a single composition that is pre-mixed prior to administration or as two separate compositions that are administered at two different points in time that are close enough to allow the ingredients to interact together once inside the body. This two phase administration of the composition would allow HMB, in either a high or low glycemic medium to be taken prior to exercise to have the anti-catabolic component of this composition inside the muscle prior to exercise and ready to prevent muscle catabolism even as exercise proceeds. This would be especially helpful for diabetic subjects where HMB in a low glycemic medium would be slowly absorbed and pre-administration could be timed to insure the HMB had time to be absorbed into the muscles prior to exercise and exert its benefit. The essential amino acids, in either a high or low glycemic medium, would be administered immediately after exercise to increase protein synthesis and the anabolic response. The decreased catabolism and increased anabolic response would give a net gain in protein synthesis and prevent delayed onset muscle soreness.

The composition of the present invention can be administered as a single core formulation or a tandem formulation, in which there is a pre-workout administration and a post-workout administration. In the core formulation, the composition comprises, based on the total weight of the composition, from about 5% to about 97% by weight of high glycemic carbohydrates, from about 1% to about 18% by weight of essential amino acids, and from about 0.5% to about 8% by weight of beta-hydroxy-beta-methylbutyrate (“HMB”). In the tandem formulation, there is a pre-workout portion and a post-workout portion. The pre-workout portion of the composition comprises, based on the total weight of the pre-workout portion, from about 5% to about 97% by weight of high glycemic carbohydrates and from about 0.5% to about 8% by weight of beta-hydroxy-beta-methylbutyrate (“HMB”). The post-workout portion of the composition comprises, based on the total weight of the post-workout portion, from about 5% to about 97% by weight of high glycemic carbohydrates and from about 1% to about 18% by weight of essential amino acids. The tandem formulation can be administered so that the pre-workout portion is taken before and/or during the workout and the post-workout portion is taken during and/or after the workout. The pre-workout portion should be taken prior to the post-workout portion.

The composition of the present invention may be administered via any route, including but not limited to orally, intraperitoneally and intravenously. Also, any salt or chelate of any of the present compounds may be used to aid absorption, e.g. HMB salts (CaHMB), essential amino acid salts (lysine-HCl). In addition, the composition can be given in all common dosage Forms including extended release dosage forms, liquid drinks, gels, paste, powders, tablets, capsules, chewable wafers, injectables, and incorporated into foods (e.g. sports bars).

The dosage ranges of the present inventions will vary depending upon the needs of the animal or human to which the composition is administered.

Some working and optimal ranges for the various components of the composition are given below in Tables 3-18.

TABLE 3
High Glycemic Sugars or Carbohydrates

| | | | |
|---|---|---|---|
| Small Animals | Working Range | 1-34 | g |
| (5 to 150 lbs) | Optimal Range | 10-20 | g |
| | Optimal Dose | 15 | g |
| Humans | Working Range | 12-70 | g |
| (60 to 300 lbs) | Optimal Range | 25-45 | g |
| | Optimal Dose | 35 | g |
| Large Animals | Working Range | 35-665 | g |
| (151 to 1200 lbs) | Optimal Range | 110-590 | g |
| | Optimal Dose | 350 | g |

TABLE 4
Essential Amino Acids

| | | | |
|---|---|---|---|
| Small Animals | Working Range | 0.2-6 | g* |
| (5 to 150 lbs) | Optimal Range | 2-4 | g |
| | Optimal Dose | 3 | g |
| Humans | Working Range | 2-12 | g |
| (60 to 300 lbs) | Optimal Range | 4-8 | g |
| | Optimal Dose | 6 | g |
| Large Animals | Working Range | 6-48 | g |
| (151 to 1200 lbs) | Optimal Range | 11-33 | g |
| | Optimal Dose | 27 | g |

*These amounts represent the sum of one or more essential L-form amino acids.
Additional D-form amino acids would be in addition to these amounts. Racemic mixtures of D, L-form amino acids would require twice the amounts shown.

TABLE 5
Beta-Hydroxy-Beta-methylbutyrate

| | | | |
|---|---|---|---|
| Small Animals | Working Range | 0.1-3 | g |
| (5 to 150 lbs) | Optimal Range | 1-2 | g |
| | Optimal Dose | 1.5 | g |
| Humans | Working Range | 1-5 | g |
| (60 to 300 lbs) | Optimal Range | 2-4 | g |
| | Optimal Dose | 3 | g |
| Large Animals | Working Range | 4-30 | g |
| (151 to 1200 lbs) | Optimal Range | 10-20 | g |
| | Optimal Dose | 15 | g |

TABLE 6
Protein

| | | | |
|---|---|---|---|
| Small Animals | Working Range | 0.4-14 | g* |
| (5 to 150 lbs) | Optimal Range | 4-8 | g |
| | Optimal Dose | 6 | g |
| Humans | Working Range | 4.8-28 | g |
| (60 to 300 lbs) | Optimal Range | 10-18 | g |
| | Optimal Dose | 14 | g |
| Large Animals | Working Range | 14-108 | g |
| (151 to 1200 lbs) | Optimal Range | 44-76 | g |
| | Optimal Dose | 60 | g |

*The numbers shown pertain to intact, partially hydrolyzed, or completely hydrolyzed protein, including any polypeptide containing one or more of the 20 amino acids that make up biological proteins.

TABLE 7
Caffeine

| | | | |
|---|---|---|---|
| Small Animals | Working Range | 0.003-0.1 | g |
| (5 to 150 lbs) | Optimal Range | 0.025-0.075 | g |
| | Optimal Dose | 0.05 | g |
| Humans | Working Range | 0.04-0.2 | g |
| (60 to 300 lbs) | Optimal Range | 0.075-0.125 | g |
| | Optimal Dose | 0.1 | g |
| Large Animals | Working Range | 0.1-0.8 | g |
| (151 to 1200 lbs) | Optimal Range | 0.15-0.55 | g |
| | Optimal Dose | 0.35 | g |

TABLE 8
L-Glutamine

| | | | |
|---|---|---|---|
| Small Animals | Working Range | 0.2-7 | g |
| (5 to 150 lbs) | Optimal Range | 2-4 | g |
| | Optimal Dose | 3 | g |
| Humans | Working Range | 3-14 | g |
| (60 to 300 lbs) | Optimal Range | 5-9 | g |
| | Optimal Dose | 7 | g |
| Large Animals | Working Range | 8-55 | g |
| (151 to 1200 lbs) | Optimal Range | 22-42 | g |
| | Optimal Dose | 32 | g |

TABLE 9
Ginger Extract

| | | | |
|---|---|---|---|
| Small Animals | Working Range | 15-475 | mg* |
| (5 to 150 lbs) | Optimal Range | 145-345 | mg |
| | Optimal Dose | 245 | mg |
| Humans | Working Range | 190-1000 | mg |
| (60 to 300 lbs) | Optimal Range | 250-750 | mg |
| | Optimal Dose | 500 | mg |
| Large Animals | Working Range | 476-4000 | mg |
| (151 to 1200 lbs) | Optimal Range | 1200-3200 | mg |
| | Optimal Dose | 2238 | mg |

*The numbers shown pertain to a 1:5 to 1:20 concentrate of ginger extract.

TABLE 10
Boswellin

| | | | |
|---|---|---|---|
| Small Animals | Working Range | 20-584 | mg* |
| (5 to 150 lbs) | Optimal Range | 148-348 | mg |
| | Optimal Dose | 248 | mg |
| Humans | Working Range | 230-1200 | mg |
| (60 to 300 lbs) | Optimal Range | 400-800 | mg |
| | Optimal Dose | 600 | mg |
| Large Animals | Working Range | 585-4675 | mg |
| (151 to 1200 lbs) | Optimal Range | 1100-3100 | mg |
| | Optimal Dose | 2100 | mg |

*The numbers shown pertain to boswellin that is standardized to 10-50% boswellic acid.

TABLE 11
Turmeric extract

| | | | |
|---|---|---|---|
| Small Animals | Working Range | 1-35 | mg* |
| (5 to 150 lbs) | Optimal Range | 10-22 | mg |
| | Optimal Dose | 16 | mg |
| Humans | Working Range | 1-100 | mg |
| (60 to 300 lbs) | Optimal Range | 20-50 | mg |
| | Optimal Dose | 35 | mg |
| Large Animals | Working Range | 35-380 | mg |
| (151 to 1200 lbs) | Optimal Range | 108-308 | mg |
| | Optimal Dose | 208 | mg |

*The numbers shown pertain to turmeric extract that is 90-95% curcuminoids.

TABLE 12
Vitamin C

| | | | |
|---|---|---|---|
| Small Animals | Working Range | 15-475 | mg |
| (5 to 150 lbs) | Optimal Range | 145-345 | mg |
| | Optimal Dose | 245 | mg |
| Humans | Working Range | 190-1000 | mg |
| (60 to 300 lbs) | Optimal Range | 250-750 | mg |
| | Optimal Dose | 500 | mg |
| Large Animals | Working Range | 476-4000 | mg |
| (151 to 1200 lbs) | Optimal Range | 1200-3200 | mg |
| | Optimal Dose | 2238 | mg |

TABLE 13
Vitamin E

| | | | |
|---|---|---|---|
| Small Animals | Working Range | 25-800 | IU |
| (5 to 150 lbs) | Optimal Range | 200-600 | IU |
| | Optimal Dose | 400 | IU |
| Humans | Working Range | 100-2000 | IU |
| (60 to 300 lbs) | Optimal Range | 400-1200 | IU |
| | Optimal Dose | 800 | IU |
| Large Animals | Working Range | 800-6200 | IU |
| (151 to 1200 lbs) | Optimal Range | 2000-5000 | IU |
| | Optimal Dose | 3500 | IU |

TABLE 14
Selenium

| | | | |
|---|---|---|---|
| Small Animals | Working Range | 10-68 | mcg |
| (5 to 150 lbs) | Optimal Range | 30-50 | mcg |
| | Optimal Dose | 40 | mcg |
| Humans | Working Range | 27-136 | mcg |
| (60 to 300 lbs) | Optimal Range | 50-90 | mcg |
| | Optimal Dose | 70 | mcg |
| Large Animals | Working Range | 68-545 | mcg |
| (151 to 1200 lbs) | Optimal Range | 206-406 | mcg |
| | Optimal Dose | 306 | mcg |

TABLE 15
Aspirin (acetylsalicylic acid)*

| | | | |
|---|---|---|---|
| Small Animals | Working Range | 25-650 | mg |
| (5 to 150 lbs) | Optimal Range | 150-500 | mg |
| | Optimal Dose | 325 | mg |
| Humans | Working Range | 100-200 | mg |
| (60 to 300 lbs) | Optimal Range | 325-975 | mg |
| | Optimal Dose | 650 | mg |
| Large Animals | Working Range | 650-5067 | mg |
| (151 to 1200 lbs) | Optimal Range | 1550-4100 | mg |
| | Optimal Dose | 2850 | mg |

*Note that human drugs may not be suitable for all animals, e.g. cats.

TABLE 16
Ibuprofen*

| | | | |
|---|---|---|---|
| Small Animals | Working Range | 10-400 | mg |
| (5 to 150 lbs) | Optimal Range | 100-300 | mg |
| | Optimal Dose | 200 | mg |
| Humans | Working Range | 100-800 | mg |
| (60 to 300 lbs) | Optimal Range | 300-600 | mg |
| | Optimal Dose | 400 | mg |
| Large Animals | Working Range | 400-3200 | mg |
| (151 to 1200 lbs) | Optimal Range | 1000-2600 | mg |
| | Optimal Dose | 1800 | mg |

*Note that human drugs may not be suitable for all animals, e.g. dogs.

TABLE 17
Naproxen*

| | | | |
|---|---|---|---|
| Small Animals | Working Range | 5-200 | mg |
| (5 to 150 lbs) | Optimal Range | 50-150 | mg |
| | Optimal Dose | 100 | mg |
| Humans | Working Range | 50-400 | mg |
| (60 to 300 lbs) | Optimal Range | 100-300 | mg |
| | Optimal Dose | 200 | mg |
| Large Animals | Working Range | 200-1600 | mg |
| (151 to 1200 lbs) | Optimal Range | 500-1300 | mg |
| | Optimal Dose | 900 | mg |

*Note that human drugs may not be suitable for all animals, e.g. dogs and cats.

TABLE 18
Acetaminophen*

| | | | |
|---|---|---|---|
| Small Animals | Working Range | 30-1000 | mg |
| (5 to 150 lbs) | Optimal Range | 250-750 | mg |
| | Optimal Dose | 500 | mg |
| Humans | Working Range | 250-2000 | mg |
| (60 to 300 lbs) | Optimal Range | 500-1500 | mg |
| | Optimal Dose | 1000 | mg |
| Large Animals | Working Range | 1000-8000 | mg |
| (151 to 1200 lbs) | Optimal Range | 2250-6750 | mg |
| | Optimal Dose | 4500 | mg |

*Note that human drugs may not be suitable for all animals, e.g. dogs and cats.

Dosages are designed to cover the spectrum of body weights or small animals to large animals, with humans in the middle. The following examples are extrapolated from the optimal dose for a 70 kg human and are used illustratively and do not limit in any way the present invention.

### EXAMPLE 1

In a preliminary investigation, eight normal healthy individuals of both sexes with an age range of 24-55 years took part in various types of aerobic or anaerobic exercise (weight training, running, kickboxing) to such a degree as to insure that delayed onset muscle soreness would ensue the following day. After the first training session, each individual immediately drank 500 ml of a post-workout supplement corresponding to Formula 7 shown in Table 1, consisting of 35 g sucrose, 6 g essential amino acids (0.65 g histidine, 0.6 g isoleucine, 1.12 g leucine, 0.93 g lysine, 0.19 g methionine, 0.93 g phenylalanine, 0.88 g threonine, 0.7 g valine), 3 g CaHMB and 100 mg caffeine flavored with raspberry-lemonade. A questionnaire was filled out over the following 24 hours post exercise. The following week, the same individuals took part in the same exercise program they had preformed the previously week for the same time and using the same intensity in their workout, but without using the present invention. Again, a questionnaire was completed over the following 24 hours post exercise. The questionnaire asked the individuals to rate how they felt both physically and mentally using a visual analogue scale (VAS). All individuals reported extreme muscle exhaustion and feeling extremely tired after both workouts. After the first workout using the post-workout supplement, all individuals reported little to no muscle soreness at approximately six and 24 hours post-exercise and most felt very good. None of the individuals used any other supplements or analgesics during the study. After the second workout where the individuals did not use the post-workout supplement all individuals reported muscle soreness at approximately six and 24 hours post-exercise and mood varied from good to bad. One individual had to use analgesics to help deal with the soreness. All individuals reported that they experienced less delayed onset muscle soreness and felt better following their workout using the post-workout supplement.

### EXAMPLE 2

A 33-year-old healthy female who was initially taking part in the exercise study in Example 1 had injured her lower back and experienced extreme pain and soreness to the degree that she required medical attention. She was administered an injectable muscle relaxer by her physician, but the pain and soreness returned after the medication wore off the following day. That evening, she did 1 hour of lower back stretching exercises followed immediately by drinking 500 ml of a post-workout supplement corresponding to Formula 1 shown in Table 1, consisting of 35 g sucrose, 6 g essential amino acids (0.65 g histidine, 0.6 g isoleucine, 1.12 g leucine, 0.93 g lysine, 0.19 g methionine, 0.93 g phenylalanine, 0.88 g threonine, 0.7 g valine), 3 g CaHMB and flavored with raspberry-lemonade. The following morning when she awoke, she experienced only minor stiffness, but all pain and soreness was resolved. She reported that her back has been better ever since. It is her opinion that the post-workout supplement allowed her to perform more physical therapy in the form of stretching exercises without further aggravation to her back injury, which contributed to her recovery.

### EXAMPLE 3

A female in her mid thirties who had suffered for a number of years with fibromyalgia after a clinical diagnosis by a physician was experiencing difficulty with her exercise regimen. She was encouraged to exercise to preserve muscle tone and strength, but could only perform exercises of such a leisurely nature to be of little benefit; otherwise severe muscle pain resulted from too much muscle use. She started to increase her exercise routine gradually and drank 500 ml of a post-workout supplement corresponding to Formula 1 shown in Table 1, consisting of 35 g sucrose, 6 g essential amino acids (0.65 g histidine, 0.6 g isoleucine, 1.12 g leucine, 0.93 g lysine, 0.19 g methionine, 0.93 g phenylalanine, 0.88 g threonine, 0.7 g valine) 3 g CaHMB and flavored with strawberry-lemonade. On one day she reported being able to walk up to 2¼ miles in 40 minutes, which left her feeling extremely tired. On her VAS questionnaire, she noted little muscle soreness at approximately six and 24 hours post-exercise and felt very good. On another day she preformed 20 minutes of exercise consisting of stretching, stair exercises, lunges, knee exercises and arm weights, which left her feeling moderately tired. On her VAS questionnaire, she indicated little muscle soreness at approximately six and 24 hours post-exercise and felt very good. It is her opinion that because of the post-workout supplement, she was able to exercise more intensely without complications such as post-exercise delayed muscle soreness and heightened muscle pain due to the fibromyalgia.

### EXAMPLE 4

A 32-year-old healthy male who had previously used a post-workout supplement corresponding to Formula 7 shown in Table 1 was given 500 ml of a post-workout supplement corresponding to Formula 7 shown in Table 2, consisting of 35 g sucrose, 3 g CaHMB, and 100 mg caffeine flavored with raspberry-lemonade immediately after his workout. The individual was not informed beforehand of the change in formulas. He reported that he did not feel the calming effect on his muscles and the feeling of general well being, which he described as being “in the zone.” He did note that his muscles were not sore at 6 and 24 hours. Therefore, it appears that the high-glycemic drink can drive the CaHMB into the muscles to improve its efficacy, but it is the synergy of both the essential amino acids and CaHMB being driven into the muscles by insulin, a well as the metabolic cascade that is induced by the carbohydrates and exercise, that gives the feeling of being “in the zone.”

### EXAMPLE 5

Two males and a female from example 1 drank a 500 ml pre-workout drink containing 3 g CaHMB, 35 g sucrose, 100 mg caffeine, and flavored with raspberry lemonade 30 minutes prior to exercise. Immediately following exercise, they drank a 500 ml post-workout drink consisting of 35 g sucrose, 6 g essential amino acids (0.65 g histidine, 0.6 g isoleucine, 1.12 g leucine, 0.93 g lysine, 0.19 g methionine, 0.93 g phenylalanine, 0.88 g threonine, 0.7 g valine), and 100 mg caffeine and flavored with raspberry lemonade. They were then asked to compare the pre- and post-workout versions of this composition to the original single formula post-workout formula. All three agreed that the two formulations taken before and after the workout gave identical results to the original formulation used in example 1 in that they did experience muscle relaxation and mental tranquility after exercise and there was no muscle soreness the following day. This example demonstrates that when the anti-catabolic component of this composition is administered prior to exercise and the anabolic component of this composition is administered after exercise, the components combine and act synergistically within the body as if when added as a single composition. Therefore, when any of the parts of these compositions are taken separately within a reasonable period of time, they combine within the body and provide the same synergy as if administered as a single composition.

### EXAMPLE 6

A 74-year old female who had suffered from a degenerated hip elected to undertake elective hip replacement surgery. During the previous year her mobility had become severely limited and she could walk no further than across the room without having to sit and rest. As a result, her physical strength diminished due to lack of activity. On about the sixth post-operative day, physical therapy was started and consisted of hip abductions, hip extensions, knee raises, knee flexions, ankle pumps, bed supported knee bends and straight leg raises. As therapy progressed, rubber bands were added to add resistance to the exercise routine. After each therapy session, the patient volunteered to consume 500 ml of a post-workout supplement corresponding to Formula 7 shown in Table 1, consisting of 35 g sucrose, 6 g essential amino acids (0.65 g histidine, 0.6 g isoleucine, 1.12 g leucine, 0.93 g lysine, 0.19 g methionine, 0.93 g phenylalanine, 0.88 g threonine, 0.7 g valine), 3 g CaHMB and 100 mg caffeine flavored with raspberry-lemonade immediately following physical therapy. As a result, at no time did the patient report soreness due to therapy whereas other hip replacement patients in her physical therapy group did report muscle soreness, especially in their thighs. On one occasion, the patient did not consume the post-workout supplement following physical therapy and as a result she reported soreness in the muscles of her legs the following day. The patient made a full recovery and progressed well through her physical therapy and rehabilitation.

### EXAMPLE 7

A two-year old male quarter horse in training for barrel racing was worked to the point of exhaustion and to where muscle soreness would ensue the following day. Immediately after exercise, 100 cc of an oral paste containing 10 g HMB, 20 g essential amino acids (3.30 g arginine, 2.20 g histidine, 2.00 g isoleucine, 3.70 g leucine, 3.10 g lysine, 0.63 g methionine, 3.20 g phenylalanine, 2.90 g threonine, 3.00 tryptophan, 2.30 g valine), 10 g glucose, 10 g sucrose, 30 g molasses, gelling agents and apple flavor, followed by one-half gallon of water containing one-cup sucrose (approximately 200 g) at room temperature. The trainer reported that typically after such training, the horse would have to rest for two days before being ready for another workout. In this case, after supplementation with this composition, the trainer was able to train the horse the following day and judged his performance to be as good as the previous days workout.

### EXAMPLE 8

In a preliminary investigation, five normal healthy individuals of both sexes with an age range of 24-55 years took part in various types of aerobic or anaerobic exercise (weight training, running, kickboxing) to such a degree as to insure that delayed onset muscle soreness would ensue the following day. After the first training session, each individual immediately drank 500 ml of a post-workout supplement corresponding to Formula 7 shown in Table 1, consisting of 35 g sucrose, 6 g essential amino acids (0.65 g histidine, 0.6 g isoleucine, 1.12 g leucine, 0.93 g lysine, 0.19 g methionine, 0.93 g phenylalanine, 0.88 g threonine, 0.7 g valine), 3 g CaHMB and 100 mg caffeine flavored with raspberry-lemonade. A questionnaire was completed one-hour following consumption of the supplement. The following week, the same individuals took part in the same exercise program they had preformed the previously week for the same time and using the same intensity in their workout, but without using the present invention. Again, a questionnaire was completed one-hour following consumption of the supplement. The questionnaire asked the individuals to rate: (1) degree of muscle/physical relaxation, (2) their perceived degree of mental tranquility, and (3) their perceived degree of general well being using a visual analogue scale (VAS). All individuals reported that they perceived a greater degree of muscle/physical relaxation, a greater feeling of mental tranquility, and a greater feeling of general well being following their workout using the post-workout supplement.

### REFERENCES CITED

The entire content of each of the following documents is hereby incorporated by reference.

### U.S. PATENT DOCUMENTS

| | | |
|---|---|---|
| 4992470 | Feb. 12, 1991 | Nissen |
| 5028440 | Jul. 2, 1991 | Nissen |
| 5087472 | Feb. 11, 1992 | Nissen |
| 5348979 | Sep. 20, 1994 | Nissen |
| 5360613 | Nov. 1, 1994 | Nissen |
| 6031000 | Feb. 29, 2000 | Nissen |
| 6090978 | Jun. 18, 2000 | McCoy |
| 6103764 | Aug. 15, 2000 | Nissen |
| 6245378 | Jun. 12, 2001 | Cavazza |
| 6248922 | Jun. 19, 2001 | McCoy |
| 6291525 | Sep. 18, 2001 | Nissen |
| 6812249 | Nov. 2, 2004 | Abraham |

### OTHER PUBLICATIONS

Biolo, G., et al. Increased rates of muscle protein turnover and amino acid transport after resistance exercise in humans. Am. J. Physiol. Endocrinol. Metab. 268: E514-520, 1995a.
Biolo, G., et al. Physiologic hyperinsulinemia stimulates protein synthesis and enhances transport of amino acids into human skeletal muscle. J. Clin. Invest. 95:811-819), 1995b.
Biolo, G., et al. An abundant supply of amino acids enhances the metabolic effect of exercise on muscle protein. Am. J. Physiol. Endocrinol. Metab. 273:E122-E129, 1997.
Brand-Miller, J., et al. The New Glucose Revolution. New York, N.Y.: Marlow & Company; 317-318.
Byrd, P., et al. Changes in muscle soreness and strength following downhill running: effects of creatine, HMB and Betagen supplementation. Med. Sci. Sports Exerc. 31:S263, 1999.
Chandler, R. M., et al. Dietary supplements affect the anabolic hormones after weight training exercise. J. Appl. Physiol. 76:839-845, 1994.
Frexes-Steed, M., et al. Role of leucine and other amino acids in regulating protein metabolism in vivo. Am. J. Physiol. (Endocrinol. Metabol.) 262:E925-E935, 1992.
Gallagher, P. M., et al. β-hydroxy-β-methylbutyrate ingestion, Part I: Effects on strength and fat free mass. Med. Sci. Sports Exerc. 32:2109-2115, 2000.
Guyton, A. C. Insulin, glucagon and diabetes mellitus. In: Textbook of Medical Physiology 6^{th} Ed. Philadelphia, Pa.: W. B. Sanders; 1981:959-972.
Jackson, N. S., et al., Nutrition and Animal Health, In: Animal Health, 3d ed., Interstate Publishers, Danville, Ill., 2000:14-17.
Jówko, E., et al. Creatine and β-hydroxy-β-methylbutyrate (RMB) additively increases lean body mass and muscle strength during a weight training program. Nutrition 17:558-566, 2001.
Knitter, A. E. et al. Effects of β-hydroxy-β-methylbutyrate on muscle damage following a prolonged run. J. Applied Physiol. 89:1340-1344, 2000.
Kraemer, W. J., Physiological adaptations to anaerobic and aerobic endurance training programs. In: Essentials of Strength Training and Conditioning, 2^{nd} ed. T. R. Baechle and R. W. Earle, Eds. Human Kinetics, Champaign, Ill. 2000:137-168.
Kreider. R. B., et al. Effects of calcium beta-hydroxy-beta-methylbutyrate (HMB) supplementation during resistance-training on markers of catabolism, body composition and strength. Intl. J. Sports Med. 20:503-509, 1999.
Nair, K. S., et al. Leucine as a regulator of whole body and skeletal muscle protein metabolism in humans. Am. J. Physiol. 263:E928-E934, 1992.
Nissen S., et al., The effect of the leucine metabolite β-hydroxy-β-methylbutyrate on muscle metabolism during resistance-exercise training. J. Appl. Physiol. 81:2095-2104, 1996.
Nissen, S. β-hydroxy-β-methylbutyrate. In: Wolinsky, I & Driskell, J. A., Eds. Nutritional Ergogenic Aids. Boca Raton, Fla.: CRC Press; 2004; 147-170.
Panton, I. B., et al. Nutritional supplementation of the leucine metabolite β-hydroxy-β-methylbutyrate (HMB) during resistance training. Nutrition 16:734-739, 2000.
Rasmussen, B. B., et al. An oral essential amino acid-carbohydrate supplement enhances muscle protein anabolism after resistance exercise. J. Appl. Physiol. 88:386-392, 2000.
Sabourin, P. J. and Bieber, L. L. Formation of β-hydroxyisovalerate from α-ketoisocaproate by a soluble preparation from rat liver. Dev. Biochem. 18:149-154, 1981.
Sparkman, D. R., Pack on the protein. New research shows how protein supplements can give you the anabolic edge. Muscle & Fitness; 55; 11:130-132, 193-194, 1994.
Sparkman, D. R., The hormones that control growth. Part 1: Insulin Starts the Growth Process for Muscles. Muscular Development, 33; 1:42-144, 183, 1996a.
Sparkman, D. R., Harness the power of glutamine to build muscle, keep fit, and avoid overtraining, Muscular Development, 33; 11:100-102, 196, 1996b.
Sparkman, D. R., HMB anticatabolic supplement increases strength and muscle mass. All Natural Muscular Development; 34; 8:164-165, 195, 203, 1997.
Tipton, K. D., et al., Nonessential amino acids are not necessary to stimulated net muscle protein synthesis in healthy volunteers. J. Nutr. Biochem. 10: 89-95, 1999.
Van Someren, K. A., et al. Supplementation with β-hydroxy-β-methylbutyrate (HMB) and α-ketoisocaproic acid (KIC) reduces signs and symptoms of exercise-induced muscle damage in man. Int. J. Sport Nutr. Exer. Metab. 15:413-424, 2005.
Vikovich, M. D., et al. β-hydroxy-β-methylbutyrate (HMB) kinetics and the influence of glucose ingestion in humans. J. Nutr. Biochem. 12:631-639, 2001.
Wagenmakers, A. J. M., et al., the metabolic fate of branched chain amino acids and 2-oxo acids in rat muscle homogenates and diaphragms. Int. J. Biochem. 17:957-965, 1985.
Whitney, E. N & Rolfes, S. R., In Understanding Nutrition, 9^{th} ed., Wadworth, 2002, pp 167-195.
Zachwieja, J. J., et al., Beta-hydroxy-beta-methylbutyrate (HMB) is produced in vivo in humans from leucine. FASEB. J. 14:A747, 2004.
Zhang, Z., et al. Change in plasma β-hydroxy-β-methylbutyrate (HMB) by feeding leucine, alpha-ketoisocaporate (KIC) and isovaleric acid (IVA) to pigs. FASEB J. 7, A392, 1993.

1. A composition for treating acute inflammation and preventing or reducing the resulting symptoms of delayed onset muscle soreness following overuse of muscles comprising:
one or more insulin mimicking or potentiating agents; and
a source of beta-hydroxy-beta-methylbutyrate.

2-62. (canceled)

63. The composition of claim 1, further comprising one or more high glycemic carbohydrates.

64. The composition of claim 1, further comprising one or more essential amino acids.

65. The composition of claim 1, wherein the source of beta-hydroxy-beta-methylbutyrate is beta-hydroxy-beta-methylbutyrate, or a precursor of beta-hydroxy-beta methylbutyrate selected from the group consisting of α-ketoisocaproate, arginine-ketoisocaproate, or leucine, salts thereof and mixtures thereof.

66. The composition of claim 1, wherein, based on the total weight of the composition, the insulin mimicking or potentiating agents range from about 0.002% to about 14%, preferably about 0.01%, and the beta-hydroxy-beta-methylbutyrate ranges from about 0.5% to about 8% by weight, preferably about 7%, and further comprising high glycemic carbohydrates in a range from about 5% to about 97% by weight, preferably about 78%, and essential amino acids in a range from about 1% to about 18% by weight, preferably about 13%, preferably in amino acid ratios that are proportional to their respective requirements in muscle protein

67. The composition of claim 63, wherein the high glycemic carbohydrates comprise any carbohydrates that are capable of eliciting an insulin response in either humans or animals.

68. The composition of claim 63, wherein the high glycemic carbohydrates comprise sugars, polysaccharides, or mixtures thereof.

69. The composition of claim 63, wherein the high glycemic carbohydrates comprise sugars selected from the group consisting of glucose, dextrose, maltose, sucrose, ribose, molasses, dehydrated cane syrup, maple syrup, honey, corn syrup, high fructose corn syrup, fruit juices, and mixtures thereof.

70. The composition of claim 63, wherein the high glycemic carbohydrates comprise polysaccharides selected from the group consisting of maltodextrins, starches, flours, whole food or food extracts and mixtures thereof.

71. The composition of claim 64, wherein the essential amino acids comprise arginine, histidine, isoleucine, leucine, lysine, methionine, phenylalanine, threonine, tryptophan, valine, salts or chelates thereof, or mixtures thereof.

72. The composition of claim 64, wherein the essential amino acids are in L-form or a racemic D, L-form mixture and may contain added D-form amino acids.

73. The composition of claim 1, wherein the source of beta-hydroxy-beta-methylbutyrate is beta-hydroxy-beta-methylbutanoic acid, calcium beta-hydroxy-beta-methylbutyrate, beta-hydroxy-beta-methylbutyrate amino acid salt, or tricreatine beta-hydroxy-beta-methylbutyrate.

74. The composition of claim 1, further comprising protein.

75. The composition of claim 74, wherein the protein is intact, partially hydrolyzed, or completely hydrolyzed.

76. The composition of claim 74, wherein the protein comprises whey protein, soy protein, egg white protein, milk caseinate protein, meat extract protein, royal jelly, any polypeptide containing one or more of the 20 amino acids that make up biological proteins, or mixtures thereof.

77. The composition of claim 1, further comprising one or more performance enhancing agents.

78. The composition of claim 77, wherein the performance enhancing agents comprise caffeine, creatine, carnitine or salts thereof.

79. The composition of claim 64, further comprising the conditionally essential amino acids L-arginine, L-cysteine, glycine, L-glutamine, L-tyrosine or mixtures thereof.

80. The composition of claim 1, further comprising one or more anti-inflammatory agents.

81. The composition of claim 80, wherein the anti-inflammatory agents comprise ginger (Zingiber officinale), boswellin (Boswellia serrata), curcumen (Curcuma longa), Harpagophytum procumbens, Humulus lupulus, Punica granatum, Tripterygium wilordii Hook F, hydroxytyrosol, pycnogenol, methylsulfonylmethane, extracts thereof or mixtures thereof.

82. The composition of claim 1, further comprising one or more anti-oxidant agents.

83. The composition of claim 82, wherein the anti-oxidant agents comprise vitamin A, beta-carotene, vitamin C, vitamin E, selenium, a-lipoic acid, glutathione, superoxide dismutase, polyphenols, anthocyanins, flavonoids, carotenoids, astaxanthin, fucoxanthins, cinnamon extracts, fruit extracts, vegetable extracts or mixtures thereof.

84. The composition of claim 1, wherein the insulin-mimicking agents comprise cinnamon, aqueous extract containing methylhydroxychalcone polymer (“MHCP”), banaba (Lagerstroemia speciosa), colosolic acid, L-ornthine alpha-ketoglutarate or mixtures thereof.

85. The composition of claim 1, further comprising one or more analgesic agents.

86. The composition of claim 85, wherein the analgesic agents comprise aspirin, ibuprofen, naproxen, acetaminophen, or any other pharmacological compound designed to reduce pain, or mixtures thereof.

87. The composition of claim 1, wherein the composition is formulated as an extended release dosage form, liquid drink, gel, powder, tablet, capsule, chewable wafer, injectable, or food supplement.

88. A kit for treating acute inflammation and preventing or reducing the resulting symptoms of delayed onset muscle soreness following overuse of muscles in a subject taking part in any physical activity, comprising:
a pre-workout composition comprising one or more insulin mimicking or potentiating agents; and beta-hydroxy-beta-methylbutyrate; and
a post-workout composition comprising optional use of one or more insulin mimicking or potentiating agents; and one or more essential amino acids, wherein the pre-workout composition is administered to the subject prior to and/or during physical activity and the post-workout composition is administered to the subject during and/or after the physical activity, and wherein the pre-workout composition is administered prior to the post-workout composition.

89. The kit of claim 88, wherein the pre-workout composition and the post-workout composition further comprise high glycemic carbohydrates.

90. The kit of claim 88, wherein the sources of beta-hydroxy-beta-methylbutyrate is beta-hydroxy-beta-methylbutanoic acid, calcium beta-hydroxy-beta-methylbutyrate, beta-hydroxy-beta-methylbutyrate amino acid salt, or tricreatine beta-hydroxy-beta-methylbutyrate, α-ketoisocaproate, arginine-ketoisocaproate, or leucine, salts thereof or mixtures thereof.

91. The kit of claim 88, wherein the post-workout composition further comprises protein, performance enhancing agents, conditionally essential amino acids, anti-inflammatory agents, anti-oxidant agents, insulin mimicking agents, analgesic agents, or mixtures thereof.

92. The kit of claim 88, wherein the insulin-mimicking or potentiating agents comprise cinnamon, aqueous extract containing methylhydroxychalcone polymer (“MHCP”), banaba (Lagerstroemia speciosa), colosolic acid, L-orthithine alpha-ketoglutarate or mixtures thereof.
